(19) United States
(12) Patent Application Publication
(10) Pub. No.: US 2021/0128393 A1
(43) Pub. Date: May 6, 2021

(54) LENGTH ADJUSTABLE CRUTCH

(71) Applicant: PORTWILLOW PTY LTD, Queensland (AU)

(72) Inventor: Alfred Michael VEGVARY, Queensland (AU)

(21) Appl. No.: 16/487,115

(22) PCT Filed: Feb. 20, 2018
(86) PCT No.: PCT/AU2018/050136

§ 371 Date: Aug. 20, 2019

(30) Foreign Application Priority Data
Feb. 20, 2017 (AU) 2017900544

(51) Int. Cl. A61H 3/02

(52) U.S. Cl. CPC A61H 3/02; A61H 3/0288; A61H 2201/0192; A61B 5/1117; A61H 2201/1215; A61H 2201/0184; A61H 2201/5069; A61H 2201/1671

(57) ABSTRACT

An adjustable length crutch including at least one support portion to support at least a portion of the weight of a user, a leg portion associated with the at least one support portion and having a foot adapted to abut the ground in use to balance the user and a length adjustment mechanism to adjust the separation between the at least one support portion and the foot to adjust the effective length of the crutch.

### FIELD OF THE INVENTION

The present invention relates to crutches and other medical aids for supporting a person's weight during therapy or rehabilitation and particularly to those which are length adjustable to allow separation of the support and the ground.

### BACKGROUND ART

Most crutches used today are adjustable to some extent so that they can be customized to fit sizes and shapes of different patients. However, many crutches are difficult to adjust, often requiring tools. Further, ranges of such adjustments for most crutches are relatively small so that hospitals, clinics and medical wholesalers and retailers must stock a spectrum of crutch sizes which can be used by small children to very tall adults, such as, 6½ foot basketball players. Maintaining such inventories is expensive. Few prior art crutches can be adjusted throughout this entire range.

Crutches which can be adjusted to accommodate wide ranges of patient sizes are described in U.S. Pat. No. 2,172,047 to Jacobucci; U.S. Pat. No. 3,034,524 to Klino; U.S. Pat. No. 3,157,189 to Farnham; U.S. Pat. No. 3,730,198 to Johnston et al.; and, U.S. Pat. No. 5,025,820 to Gamper. An adjustable and collapsible crutch described by Jacobucci (U.S. Pat. No. 2,172,047) is constructed primarily of metallic tubing and tubing joints which are coupled together to generally form two sections which can be slid relative to one another. One of these sections A has openings along a length thereof, while the other section B has what appear to be spring-loaded metallic bolts which are driven into the openings by springs, but which can be pulled out of the openings by pinching together handles attached to the bolts. Although the Jacobucci adjustable and collapsible crutch has many advantages over normal wooden crutches, it appears that it would be too heavy and expensive for general use. In this regard, it is thought that the members of this crutch would have to be constructed of a strong metal, such as steel, in order to have the necessary strength and durability for continuous use.

It will be clearly understood that, if a prior art publication is referred to herein, this reference does not constitute an admission that the publication forms part of the common general knowledge in the art in Australia or in any other country.

### SUMMARY OF THE INVENTION

The present invention is directed to a length adjustable crutch, which may at least partially overcome at least one of the abovementioned disadvantages or provide the consumer with a useful or commercial choice.

With the foregoing in view, the present invention in one form, resides broadly in an adjustable length crutch including at least one support portion to support at least a portion of the weight of a user, a leg portion associated with the at least one support portion and having a foot adapted to abut the ground in use to balance the user and a length adjustment mechanism to adjust the separation between the at least one support portion and the foot to adjust the effective length of the crutch.

The crutch of the present invention is particularly adapted to be adjustable in order to allow quick and easy length configuration for users of different heights. The crutch of the present invention may also assist a user of limited mobility when standing and sitting.

The crutch of the present invention is particularly well adapted to use in therapeutic or rehabilitation situations. Typically, crutches are used to assist the mobility of users whilst they are recovering from injury or treatment for medical conditions or surgery. As such, they are normally a temporary aid which is dispensed with once the user has recovered their mobility. In certain situations, crutches are provided for long-term use.

In the context of the present invention, the term “crutch” is considered to include other mobility aids such as walking sticks, walking frames and the like, and is not intended to be limiting.

The crutch of the present invention and preferably includes an upper assembly and a lower leg portion. The upper assembly will typically include the at least one support portion and at least one elongate strut member which will typically form a majority of the height of the crutch.

As mentioned above, the at least one support portion of the crutch of the present invention will preferably form part of the upper assembly of the crutch. The at least one support portion may be provided as a support for a user's hand, underarm or shoulder, forearm or any combination of these. The type and configuration of the support portion(s) provided will typically depend upon the configuration of the crutch itself.

The least one support portion will typically be arcuate in cross-section as the portion will normally abut a part of a person's body and as such, will normally be rounded for comfort. The least one support portion may also be or include a resilient portion in order to minimise discomfort during periods of use.

As mentioned above, the type and configuration of the support portion(s) will typically depend upon the configuration of the crutch itself. There are two main crutch configurations.

In the first main crutch configuration, the upper assembly of the crutch includes a pair of spaced apart strut members with a shoulder or underarm rest at an upper end and extending between the strut members. The underarm rest will normally be fixed and will also function to space the strut members at the upper end. A hand support portion will typically be provided extending between the strut members approximately at arm's length downwardly from the underarm rest. Typically, the hand support portion will be temporarily fixed relative to the strut members, but the hand support portion will be movable in order to adjust in the spacing between the hand support portion and the underarm rest.

The hand support portion preferably has engagement means such as shear pins for selectively engaging openings in strut members in order to temporarily fix the position of the hand support portion. Normally a number of openings are provided in order to adjust the position of the hand support portion.

According to this configuration, the strut members of the upper assembly typically taper towards each other at a lower end. Normally, a guide portion is provided at or adjacent the lower end of the upper assembly in order to receive and/or guide the leg portion.

According to the second main crutch configuration, a single strut member is provided with a forearm support portion located at or adjacent and at the end and thereof. The forearm support portion is typically fixed relative to the strut member. The strut member is normally provided with at least a hollow portion at or adjacent the lower end. Typically, the strut member is entirely hollow.

A hand support portion is normally provided extending from the strut member approximately at arm's length downwardly from the underarm rest. Typically, the hand support portion will be temporarily fixed relative to the strut members, but the hand support portion will be movable in order to adjust in the spacing between the hand support portion and the underarm rest.

According to the second crutch configuration, the leg portion is normally receive at least partially within the hollow strut member and is at least temporarily fixed relative thereto.

According to both of the above configurations, the leg portion typically includes an elongate member which is at least temporarily securable relative to the upper assembly in order to at least temporarily fixed the length of the crutch or more particularly the distance between the foot portion of the leg and of the least one support portion. The leg member may be hollow or solid depending upon material and strict requirements. For example, the leg member may be manufactured from a metal or other material providing sufficient strength.

The leg portion will typically have a resilient foot in order to minimise slippage of the crutch in use. The foot also preferably increases the footprint area to maximise group.

The leg member may be of any shape but typically will be round or circular in cross-section.

The adjustable length crutch of the present invention also includes a length adjustment mechanism to adjust the separation between the at least one support portion and the foot to adjust the effective length of the crutch.

The length adjustment mechanism of the present invention is preferably provided in one of two main preferred forms.

According to the first preferred form, the length adjustment mechanism has a threaded or helical configuration and the components of the length adjustment mechanism are therefore adapted for this purpose. In particular, the components of the length adjustment mechanism of the first preferred form include a threaded member, drive means to rotate the threaded member in at least one, and typically both directions, and a telescopic leg portion associated with the threaded member and which extends and retracts relative to the upper assembly upon rotation of the threaded member by the drive means.

Typically, the drive means will be provided in association with the upper assembly. Further, a lower part of the upper assembly may be provided with a guide portion to guide and/or stabilise the leg portion or threaded member during extension, retraction and in use.

Typically, the drive means may be associated with a further guide means. The drive means will typically be a lightweight motor, preferably an electric motor which is battery-powered, although other types may be used. Activation of the drive means will typically cause rotation of the threaded member and may do so in any mariner. The electric motor (and/or battery) may be positioned in any location on the crutch, for example, in association with the further guide means or in/on the underarm support.

The threaded member will typically be an elongate rod with at least one threaded portion extending at least partially, and preferably completely, over the length of the rod. The rod will typically be circular cross-section, but the shape is not essential.

According to an alternative embodiment, the drive means may be associated with a shaft with a shaped, typically threaded, portion adapted to facilitate extension and retraction of a telescopic leg member through rotation of the shaft. According to this embodiment, at least a portion of each component of the telescopic leg member may be threaded and associated with a shaft in order to extend and retract.

Each of the components of the telescopic leg member may be provided with an internally threaded portion adapted to engage with the threaded rod or shaft in order to extend and retract. Typically, extension of the innermost telescopic member is actuated initially by rotation of the threaded rod or shaft relative to the respective internally threaded portion. Once the innermost telescopic member has reached the limit of its movement, the further rotation or shaft or threaded member may disengage from the internally threaded portion of the innermost member and continued rotation will then extend the next innermost telescopic member and so on until the telescopic leg member reaches the desired length or the limit of its extension. Retraction of the telescopic leg member will preferably occur in the opposite manner.

According to the second preferred form, the length adjustment mechanism includes one or more rams associated with an extendable and retractable scissor drive assembly which is in turn associated with a leg member. According to this embodiment, the leg member will typically be of fixed length and be extended and retracted to the desired position by the scissor drive assembly. The hydraulic rams will typically force the extension and retraction of the scissor drive assembly.

Typically there will be a pair of hydraulic rams, but the rams may be pneumatic or the like. Each of the hydraulic rams will typically be mounted in a pivotal fashion to a cross piece provided in association with the upper assembly. The hydraulic rams are each typically associated with a cross-piece and a portion of the scissor drive assembly.

The scissor drive assembly will typically include a plurality of pairs of arm members, the arm members of each pair typically connected to each other approximately centrally along the length. Each end of each arm member is then typically attached to an end of an arm member of an adjacent pair, which are similarly connected to each other. The first pair of arm members (the uppermost pair) will normally be associated with one of the rams.

There may be an intervening pivotably mounted plate means or the rams may attach directly to an end of each of the arm members. Provision of a number of pairs of arm members allows the extension and retraction of the leg member through the scissor action and control over the degree of extension and retraction of the scissor drive assembly will preferably act to control the separation of the foot and the at least one support portion.

The last pair of arm members (the lowermost pair) will normally be associated with the leg member. One or more intervening members may be provided.

The rams of this configuration may be provided between a pair of cross-piece members which are attached to or interposed between strut or shaft members provided as a part of the upper assembly.

The extension and retraction (and typically the speed of extension and retraction) of the leg member will typically be actuated using an actuation means in communication with the drive needs. Normally the actuation means will be a depressible or button or switch, but other components may be included in the actuation means such as speed controls. Normally, the actuation means will be associated with a hand support portion so as to be conveniently located for a user when the crutch is in use. Preferably, the actuation means is located on the hand support portion. It is anticipated however, that the actuation means may be located remotely from the hand support portion in order to minimise the chance of accidental activation of the length adjustment mechanism. If located remotely, the actuation means is preferably spaced such that the actuation is unlikely to be accidental but should be within easy reach of the user without removing their hands from the support portion.

The present invention in one form, resides broadly in a length adjustable crutch which can be used as a support by a user; the crutch including a frame having a length and which includes a support portion at a first end of the frame and a leg portion which is positioned at an opposed, second end of the frame and which can be longitudinally moved between a retracted position and an extended position so that the length of the frame can be adjusted; and a length adjustment mechanism; and wherein the length adjustment mechanism can be configured so that the leg portion is moved at least to the extended position with a predetermined level of force.

In an alternative embodiment, the invention resides in a length adjustable crutch which can be used as a support by a user; the crutch including
a) a frame including a support portion at a first end of the frame
b) a leg portion which is positioned at an opposed, second end of the frame and which can be longitudinally moved between a retracted position and an extended position to adjust the effective length of the crutch; and
c) a length adjustment mechanism including a fluid drive means to extend the leg portion by expansion of a fluid maintained under pressure and an energy capture system to capture at least a portion of the energy used to retract the leg portion to pressurise the fluid.

The crutch of the present invention is particularly adapted so that a length of the crutch can be adjusted by driving the leg portion downwards. i.e. towards the ground which supports the crutch, thereby pushing a user of the crutch upwardly. Thus, the extensioning of the crutch assists the user moving from a sitting position to an upright or standing position with minimal assistance required from another person, for example a carer.

The crutch of the present invention can be used in therapeutic or rehabilitation situations to improve the mobility of users whilst they are recovering. Although in these situations the crutch is normally a temporary aid, it is possible for the crutch of the present invention to be used as a long-term aid by users which have a permanent disability.

In the context of the present invention, the term “crutch” is considered to include other mobility aids such as walking sticks, walking frames and the like, and is not intended to be limiting.

Any suitable length adjustment mechanism can be used which allows the length adjustment mechanism to be set to cause longitudinal movement of the leg portion with a predetermined level of force. For example, the mechanism may use a gas, which may be drawn from a compressed source, or a liquid to drive one or more components of the mechanism thereby to cause longitudinal movement of the leg portion. The length adjustment mechanism may embody any suitable configuration of two or more components, for example a piston and sleeve configuration. The leg portion may be connected to any component of the mechanism thereby to cause the leg portion to move longitudinally. For example, the leg portion may be connected to the piston which is caused to move along the sleeve or to the sleeve which is caused to move along the piston. Alternatively, in a different example, the fluid mechanism may use a combination of a fluid and a biasing member such as a spring to cause the leg portion to move longitudinally.

In another example of the invention, the length adjustment mechanism may combine a fluid arrangement and a mechanical arrangement which is driven by operation of the fluid arrangement. For example, the mechanical arrangement may function on a thread or helical configuration so that the components of the mechanical arrangement are adapted to suit this configuration. The components of the mechanical arrangement may include a threaded assembly which is caused to move by the operation of the fluid arrangement so that the movement of the threaded member causes longitudinal movement of the leg portion. In one example of the invention, the threaded assembly may be a lead screw, ball screw or gear trains so that radial motion of the threaded assembly is translated into linear motion (longitudinal movement) of the leg portion. For example, when a lead screw configuration is used, the fluid arrangement may be associated with a shaft of the mechanical arrangement so that rotation of the shaft by the fluid arrangement causes movement of a nut of the mechanical arrangement so that the leg portion, which is associated with the nut, is caused to move longitudinally. Alternatively, the nut is kept stationary so that rotation of the shaft by fluid arrangement causes movement of the leg portion, which is associated with the shaft, to move longitudinally. It should be noted that the mechanical arrangement can be adapted into different configurations (i.e. the nut is caused to move or the shaft is caused to move) and the examples discussed are merely illustrative and should not be seen as limiting.

Alternatively, in a different embodiment of the invention, the mechanical arrangement includes a scissor drive assembly which is associated with the leg portion and which is caused to move, i.e. extend or retract, through the operation of the fluid arrangement. In one example of the invention, the fluid arrangement additionally includes at least one ram which causes the scissor drive assembly to extend or retract. The at least one ram can be associated with the scissor drive assembly in any suitable way so that operation of the ram causes the scissor drive assembly to extend or retract. For example, the at least one ram can be associated with a cross piece of the scissor drive assembly.

The leg portion may be associated with the length adjustment mechanism in any appropriate way to achieve longitudinal movement of the leg portion during operation of the length adjustment mechanism. For example, the leg member may be pushed, drawn, or entrained with a component of the length adjustment mechanism, for example a piston, pushrod, or sleeve. The leg portion may further be secured to a component of the length adjustment mechanism using any suitable technique such as appropriate fasteners, rivets, welding, or the like so that movement of the component of the length adjustment mechanism can be transferred to the leg portion.

The length adjustment mechanism is such that the amount of force exerted by the length adjustment mechanism on the leg portion can be adjusted. In one embodiment of the invention, when a pressurised gas is drawn from a compressed source, a delivery mechanism of the length adjustment mechanism can be adjustable thereby to select the amount of force being generated in one or more components of the length adjustment mechanism moved by the pressurised gas. In a further embodiment of the invention, possibly with an aim to reduce the volume of pressurised gas required to cause the leg portion to move longitudinally with a predetermined force, a biasing member such as a compression spring can be used to exert a force in addition to that of the pressurised gas on the one or more movable components of the length adjustment mechanism so that the combination of the biasing force of the compression spring and that of the pressurised gas achieves the predetermined level of force which is to be exerted on the leg portion during operation of the length adjustment mechanism.

In one embodiment of the invention, the length adjustable crutch includes a control assembly with which the operation of the length adjustment mechanism can be controlled. The control assembly may include a handle, an actuation member such as a mechanical or electrical switch or rocker switch or a valve, and an arrangement for connecting the actuation member to the length adjustment mechanism. In one example of the invention, the actuation member is a valve which causes operation of the length adjustment mechanism by connecting the compressed source of gas to the movable components of the length adjustment mechanism. In another example of the invention, the actuation member is a switch which, once triggered, causes a liquid to be pumped to the movable components of the length adjustment mechanism so that longitudinal movement is caused in the leg portion.

The control assembly may further include a suitable source of energy, which may be rechargeable, with which the length adjustment mechanism can be operated. In one example of the invention, the control assembly may have a suitable adapter to allow the energy source to be recharged using sources of electricity having different voltages such as a mains voltage of 240 V or a motor vehicle voltage of 12 V. Alternatively, a separate adapter may be supplied with or separately from the crutch with which the source of energy can be recharged.

It is to be understood that the examples referred to should not be seen as limiting and that other components and configuration of components can be used in the length adjustment mechanism and control assembly to cause longitudinal movement in the leg portion.

The length adjustment mechanism may be associated with the frame in any suitable way so that operation of the length adjustment mechanism does not cause it to become detached from the frame. For example, the length adjustment mechanism may be secured by using suitable fasteners, clips, rivets, welding or the like to securely connect the length adjustment mechanism to the frame. Alternatively, in another example, the mechanism used to secure the length adjustment mechanism to the frame can be such so that the length adjustment mechanism is detachable from the frame when required, for example when a component of the length adjustment mechanism needs to be replaced or repaired. In another example of the invention, the length adjustment mechanism is secured to the frame at a position which is between the leg portion and the support portion.

The control assembly may also be associated with a frame in any suitable way so that the control assembly is secured to the frame so that it does not become inadvertently detached from the frame during normal use of the crutch or even during moderately rough use such as when the crutch is accidentally dropped. The control assembly may further include a suitable cover which provides a neat finish to the crutch once the control assembly and the length adjustment mechanism has been secured to the frame.

The support portion may be pivotally movable relative to the foot portion so that the length of the frame can be adjusted. For example, the support portion can be moved between an unfolded position and a folded position so that folding of the support portion to the folded position causes the length of the frame to be reduced.

The frame may include a feature such as a lock assembly which allows the support portion to be locked in the unfolded position. Alternatively, the lock assembly may be such that the support portion can be locked in the folded and unfolded positions. The support portion may further include an end member which can be moved relative to the leg portion so that a length of the support portion can be adjusted. Any particular technique can be used to facilitate movement of the end member relative to the leg member. In one example, the support portion can be telescopically extended so that a position of the end member on the support portion or the positioning of the end member relative to the leg portion is adjustable. The telescopic extension of the support portion also may be such that the end member can be locked at any selected position or only at a number of predetermined positions. In the latter instance, the support portion may be configured to include suitable locks, for example spring-loaded pins, which secure the end member at the predetermined positions.

The frame may be constructed in any suitable way so that, during operation of the length adjustment mechanism, the frame provides the crutch with sufficient rigidity to allow for operation of the length adjustment mechanism without damaging the frame. In one example of the invention, the frame is constructed from a number of tubular members which are secured to each other using any suitable technique such as suitable fasteners, rivets, welding or the like.

The frame may have a first part which consists of two parallel, spaced apart tubular members between which the length adjustment mechanism is mounted and from which the leg portion is movable between the extended and retracted positions. The frame may further have a second part which consists of two diverging tubular members which extend from the first part so that a distance between the two tubular members increases to a maximum at a remote end of the second part. This may allow for the control assembly to be mounted to the second part so that the components of the control assembly can be nestled between the two tubular members of the second part. The cover of the control assembly may be suitably shaped to fit over the second part so that the components fitted to the second part are at least partly concealed. The handle of the control assembly may be fitted to the second part at any suitable position along a length thereof so that the handle and associates components fitted to the handle, for example the actuation member, are easily accessible by the user. In one example of the invention, the handle is fitted to the second part in proximity to the remote end of the second part and the support portion is pivotally attached to the remote end of the second part for movement between the folded and unfolded positions. The remote end of the second part may also carry a feature or lock assembly which allows the support portion to be locked in the folded position and optionally in the unfolded position.

The length adjustment mechanism may be such so that the leg portion extends from a component of the length adjustment mechanism, for example the sleeve, so that movement of the piston along the sleeve during operation of the length adjustment mechanism causes the leg portion to be moved also along the sleeve, for example, from the retracted position to the extended position.

The length adjustment mechanism may be operable to cause the leg portion to move from the retracted position to the extended position and from the extended position to the retracted position. Alternatively, the length adjustment mechanism may be operable to cause the leg portion to move only from the retracted position to the extended position and to allow the user to cause the leg portion to move from the extended position to the retracted position. For example, the control assembly may be operable, for example by including a second actuation member or only one actuation member which can perform a dual function, to allow the user to push, typically by leaning more heavily on the crutch, the leg portion from the extended position to the retracted position. In this example, the control assembly or length adjustment mechanism should be such so that the leg portion is substantially kept in the retracted position. This can be achieved in any suitable way such as by preventing the piston to be moved, or creep, along the sleeve by sealing off a chamber formed between the piston and a base of the sleeve thereby reducing the likelihood of the piston inadvertently progressing by a substantial extent towards a mouth of the sleeve. Alternatively, the piston can be mechanically locked such as by using a catch mechanism which allows the leg portion, when in the retracted position, to be secured to the mouth of the sleeve.

In one form of the invention, the length adjustment mechanism and control assembly is such that the leg portion can be kept substantially at a selected position when the leg portion has been moved partially from the retracted position to the extended position. As an example, the mechanical arrangement can be such so that the leg portion is locked in a selected position as a result of mating surfaces of the mechanical arrangement. In one example, when the mechanical arrangement is in the form of a thread assembly, the interlocking parts of the threaded assembly may prevent longitudinal movement either towards the retracted position or further towards the extended position. Consequently, if further longitudinal movement of the leg portion is required, the fluid arrangement needs to be activated to cause operation of the threaded assembly to thereby cause longitudinal movement of the leg portion. In a further example, the scissor drive assembly can be locked into position by the at least one ram so that the at least one ram prevents further movement of the leg portion towards the extended position as well as reverse movement of the leg portion towards the retracted position.

In another embodiment of the invention, the length adjusting mechanism is such so that when the user pushes the leg portion towards the retracted position, some energy used in moving the leg portion from the retracted position to the extended position, is recovered. For example, the fluid arrangement may be operable by the control assembly so that as the leg portion is moved towards the retracted position, if a gas was used to cause longitudinal movement of the leg portion, some or all of the gas drawn from the source of fluid is pushed back into the source of fluid so that this source is at least partly replenished or at least partly re-pressurised. Alternatively, when a liquid was used to cause longitudinal movement of the leg portion, the liquid being pushed back to the source of fluid could be circulated past a energy generating means, for example a small electrical generator, so that electrical energy is generated by the movement of the fluid to the source of fluid. In this way, some of the electrical energy spent moving the leg portion at his body towards the extended position.

In another example of the invention, the handle may further have a first actuation member which is accessible from an upper side (taken to be the side between the handle and the end member of the support portion) and a second actuation member which is accessible from an opposed, underside of the handle. In a further example, the handle may have additionally a third actuation member which is accessible from the upper side of the handle but from an end of the handle which opposes an end of the handle which carries the first actuation member.

The frame can be manufactured from any suitable material which allows the frame to be durable and rigid in construction but reduces the weight of the frame once assembled. For example, material such as aluminium, suitable composite metals, or a plastic material can be used.

According to another form, the invention resides broadly in a length adjustable crutch which can be used as a support by a user and which includes a frame having a length and includes a support portion at a first end of the frame and a leg portion at an opposed, second end of the frame; the support portion can be pivoted relative to the leg portion between an unfolded position and a folded position; the leg portion being movable between a retracted position and an extended position; and a length adjustment mechanism which can be operated to move the leg portion at least from the retracted position to the extended position; and wherein the frame can be adjusted between a compact configuration in which the support portion is in the folded position and the leg portion is in the retracted position, and an operative configuration at which at least the support portion is in the unfolded position.

According to a further form, the invention resides broadly in a length adjustable crutch which can be used as a support by a user, the crutch including a gripping portion and a leg portion; a mechanism which causes the leg portion to move longitudinally to an extended position from a retracted position thereby adjusting the length of the crutch; and a control assembly through which the mechanism can be operated; and wherein the mechanism uses a fluid to cause longitudinal movement of the leg portion between the retracted and extended positions.

Typically the fluid will be a hydraulic or pneumatic fluid.

The gripping portion of the crutch of the preferred embodiment will typically be substantially L-shaped. Preferably, there will be a first portion to be received and held at a handle by a user, in the user's palm with the user's fingers and thumb wrapped thereabouts.

Preferably, the handle will be located relative to a user's palm such that the handle can support the thenar and hypothenar eminences of the user's hand with the user's fingers located underneath the handle portion on one side of the handle portion and user's thumb wrapped about the handle portion from the opposite side of the handle portion. The handle portion will therefore be shaped accordingly and may be contoured.

Typically, any actuator switches are located on an underside of the handle. Preferably, at least one actuator switch is provided and this actuator switch is typically recessed into the handle in order to prevent accidental actuation. Any actuator switches will typically be located at a forward or free end of the handle portion in order to be operated by a user's forefinger or thumb.

A toggle switch is the preferred actuator switch. Typically, a toggle switch is provided which is biased into a central, inactive position and which is capable of movement to either or both of the left and right actuation positions. According to a particularly preferred embodiment, movement of the toggle switch to the left (or right) will actuate extension of the crutch and movement of the toggle switch in the opposite direction will allow retraction. In the retraction position, retraction may not be driven but it may be that actuation of the switch simply unlocks the leg, allowing a user to apply force to the leg in order to force retraction.

Typically, when the leg is extended, and the toggle switch is released, the mechanism will typically automatically lock the leg in the extended position. A separate locking switch may be provided or alternatively, the locking may be disengaged by movement of the toggle switch in the retracted direction by a predetermined distance.

As mentioned above, the handle portion will typically have a forward or free end. The opposite end of the handle portion will typically be provided with a depending portion. The depending portion will preferably be angled relative to the handle portion. The angle will typically be approximately 90°, but will preferably be slightly less than 90°.

The depending portion will preferably be at least partially hollow.

The gripping portion will typically be manufactured as a unitary member but may be a multipart construction in certain embodiments. The gripping portion will also preferably be shaped for comfort of the user particularly as a majority of the user's body weight may be supported by the gripping portion during operation.

Preferably, an opening will be provided into the depending portion. This opening will preferably be substantially coaxial with the depending portion through the upper end of the handle portion or the upper wall or end of the depending portion. A cover member may be provided for this opening. Preferably, the cover member will be attached to the gripping portion so that it is not lost when removed from the opening.

The leg portion may be of any shape or configuration. However, typically an outer leg portion and an inner leg portion are provided. Preferably, the outer leg portion is an upper leg portion located closer to the gripping portion then the inner, lower leg portion. Each of the inner and outer leg portion will typically be of approximately the same length. Multiple, nesting leg portions can be provided.

The leg portion will typically have a telescoping configuration. The leg will preferably be cylindrical in shape although advantages maybe gained from providing a rectangular leg or a leg with vertices in order to minimise rotation of the upper leg portion relative to the lower leg portion.

According to a preferred embodiment, a control housing will be provided between the gripping portion and the leg portion. Preferably, the upper leg portion is attached to or mounted to a lower side of the control housing allowing the mechanism within the control housing to drive extension of the leg portion. Preferably, the upper leg portion is received at least partially within an annular mounting collar.

The lower leg portion may be provided with a shock absorber or similar. A shock absorber may be provided at a lower end of the lower leg for absorption of shock when the leg portion is extended and the crutch is in use, or alternatively, a shock absorber may be provided to buffer the final movement of the lower leg wherein the lower leg is being retracted.

A foot or similar is preferably provided at the lower end of the lower leg portion. In one preferred embodiment, the foot includes a foot platform with a large effective area. A plurality of arms typically extend from the foot platform and four arms are preferred. The foot platform is typically a planar plate or similar with the arms extending therefrom. Preferably, the arms are arcuate, extending laterally and then downwardly. Normally, non-slip cups, pads or similar may be provided at the lower end of each of the arms in order to minimise the chance that the crutch can slip while in use.

Preferably, the extendable leg portion is mounted to one side of the foot platform, adjacent one side edge. This will typically allow the support to be provided closer to the user's body than if the leg portion was more centrally mounted on the platform. According to a preferred embodiment, the leg portion is mounted to the platform approximately centrally along the platform and adjacent one side edge.

In this embodiment, the crutch includes a mechanism to cause the leg portion to move to the extended position from a retracted position thereby adjusting length of the crutch. The control mechanism is typically contained within a control housing which is a central component of the crutch located typically relative to the gripping portion has discussed above.

The control housing will typically extend forwardly beneath the handle portion of the gripping portion approximately as far as the free end of the handle portion. Accordingly, the length of the control housing is preferably coaxial with the handle portion and located beneath the handle portion. Typically, the control housing is of minimal width, again approximating the width of the handle portion.

The control housing typically houses the actuator to cause extension of the leg such that when the actuator switches operated into either the extension or retraction position, this allows a drive system to move or drive the movement of the leg as required.

Typically, the drive system will be either hydraulic or pneumatic. Therefore, when the actuator switch is switched to the extension position, electrical power provided from a preferred the battery (which can be rechargeable or replaceable) within the control housing is provided to the pump in order to drive a fluid to extend the lower leg relative to the upper leg.

Once the limit of extension is reached, a lock can preferably engage either automatically or be engaged manually by the user. Once the lock has been engaged, manual disengagement is preferably preferred.

When the actuator is switched to the retraction position, then the pump can either operate in reverse at a given (adjustable) speed to reduce the length of the crutch at a controlled speed or in a controlled manner or alternatively, the lock will be disengaged and the user can be required or able to exert force on the handle portion to drive retraction of the leg.

In an alternative form, the drive system can be a screw drive system. In a preferred embodiment, a toggle switch will be provided with an “extend” position and a “retract” position on either side of a home or “locked” position. Operation of the toggle switch activates an onboard electrical motor to rotate a drive cog. The motor is typically powered by a battery o similar, preferably a rechargeable on board battery. Preferably the drive cog will mount a closed loop drive belt, preferably a toothed drive belt. A driven cog is preferably provided adjacent to the drive cog and separated therefrom, connected by the drive belt. Preferably the driven cog is larger in diameter than the drive cog which will increase the torque available at the driven cog. The driven cog will preferably be a toothed cog. The driven cog preferably rotates an upper portion of a threaded rod. The upper portion of the threaded rod is preferably not threaded (allowing for guided rotation) but the bulk of the length of the threaded rod is threaded. A lower portion of the threaded rod is provided with a guide which is fixed to the threaded rod and abuts an inner surface of the hollow leg tube.

The threaded rod is preferably mounted within a hollow leg tube of fixed length which extends from the handle and control assembly. A hollow extendible leg is mounted about the threaded rod and within the hollow leg tube. The hollow leg tube is preferably made of metal. The hollow leg tube is preferably circular in cross-section.

The hollow extendible leg is preferably provided with a guide boss at an upper end that is mounted about the threaded portion of the threaded rod and at least a portion of the guide boss abuts an inner surface of the hollow leg tube. A bore is provided through the guide boss and the bore is preferably threaded to engage with the threaded portion of the threaded rod. Rotation of the threaded rod will therefore preferably cause translation of the guide boss along the threaded rod which extends and retracts the hollow extendible leg.

A receiving tube is also preferably provided in the handle portion accessible using the opening described above. When the preferred cover is removed, a lower end of a forearm support can be inserted into the receiving tube. The receiving tube is attached to a housing that is attached to the hollow leg tube and surrounds the guide boss and the upper end of the drive mechanism. The handle portion of the device is also attached to the receiving tube. The receiving tube is preferably made of metal in order to provided additional strengthening and rigidity to the device allow significant forces to be transmitted to the leg.

Preferably, the speed at which extension occurs is adjustable and similarly, the speed at which retraction occurs may be adjustable as well. Further, the speed may be variable with a faster extension (or retraction) at the beginning of the movement with the movement slowing towards the end of the stroke, in one or both directions. This can be achieved by changing the fluid flows if pneumatic or hydraulic or provided a threaded portion of a different pitch at the lower and/or upper end of eth threaded rod in a threaded or screw drive assembly.

As mentioned above, the mechanism will typically be a hydraulic or pneumatic pump or ram system. Actuation of the operation will preferably result in extension and/or retraction using a working fluid which will typically be pumped either into or out of a closed volume within the leg in order to drive extension and/or retraction. The type of mechanism used to extend and/or retract the leg is not vital but the fact that it is a fluid based drive system is important as it provides a number of advantages over threaded drive systems.

The crutch of the preferred embodiment may include an optional support which is attachable to the crutch to provide additional support or stability. Preferably, the support will be attached to the crutch utilising an extension portion with one end of the extension portion attached into or relative to the opening in the handle of the gripping portion (once the cover has been removed). Typically, a part of the extension portion will extend down into the depending portion of the gripping portion for stability.

The periphery of the opening in the gripping portion may be provided with one or more deformable rings or members located inside the opening and extending thereinto in order to abut the extension portion to hold the extension portion in the opening. Typically, the deformable rings or members will be resilient and will require force to deform during insertion (and removal) of the extension portion into (and from) the opening.

A locking assembly or component may be provided in order to releasably lock the extension portion within the opening.

The extension portion may be a multipart portion. Typically, the extension portion will be length adjustable and a telescoping adjustment is preferred. Any length adjustment mechanism may be provided with a pin for location in aligned or alignable openings in one or more of the preferred telescoping components of the extension portion, being preferred for simplicity. The pin can be a depressible or spring loaded pin.

The extension portion may be provided with a collar or cuff at an upper end to receive a user's forearm or alternatively, a pad or similar could be provided to be received in a user's armpit area.

The crutch including the leg and the extension portion will preferably be manufactured of lightweight but strong materials. Preferred materials include aluminium, carbon fibre or the like.

The crutch of the present invention will also include other safety apparatus connected to the power source of the crutch. For example, the crutch of one embodiment will preferably include an alarm actuator with an associated communications pathway to allow the operator of the crutch to alert others in an emergency. The actuator will typically be a button or switch or similar and actuation will preferably cause an alarm to be issued or to transmit a signal to a remote locations. In some cases, the alarm will be sounded at the remote location or alternatively, triggering the actuator may cause a nominated telephone number to be dialed and/or a message indicating that the crutch operator needs assistance.

The crutch may be provided with a unique identifier allowing identification of the operator if the actuator is triggered. The crutch may further be provided with a positioning device such as a GPS receiver in order that the location of the crutch can be made at any time and particularly if an alert or alarm is triggered.

The crutch may include a actuator that is manually and/or automatically actuated. The crutch may be provided with an attitude sensing device which may actuate an alarm or signal should the crutch be placed in an angle that indicates that the person has fallen. Preferably there will be a manual deactivation of this functionality such that the operator can disable the attitude sensor trigger as required.

The crutch may include at least one photovoltaic cell in order to provide power to the crutch or any on board battery.

The crutch may include may include at least one light, preferably directed forwardly in order that an operator can use the light to see or to signal for help. The light is preferably manually actuable.

### BRIEF DESCRIPTION OF THE DRAWINGS

Various embodiments of the invention will be described with reference to the following drawings, in which:

FIG. 1A is a front view of an adjustable length crutch according to one embodiment of the present invention in the extended condition.

FIG. 1B is a front view of the adjustable length crutch as illustrated in FIG. 1A in the contracted condition.

FIG. 2A is a front view of an adjustable length crutch according to a second embodiment of the present invention in the extended condition.

FIG. 2B is a front view of the adjustable length crutch as illustrated in FIG. 2A in the contracted condition.

FIG. 3 is a perspective view of the adjustable length crutch as illustrated in FIG. 2A.

FIG. 4 is a perspective view of a length adjustable crutch, according to the invention, in a compact configuration.

FIG. 5 is a perspective view of the length adjustable crutch showing a leg portion of a frame of the crutch being moved to an extended position.

FIG. 6 is a perspective view of the length adjustable crutch which has been adjusted to an operative configuration.

FIG. 7 is a perspective view of a variation of the length adjustable crutch of FIG. 5 and showing a control assembly fitted to the frame.

FIG. 8 is a perspective view of the length adjustable crutch of FIG. 6 to show more clearly a cover used to conceal part of the control assembly.

FIG. 9 is a perspective view, on an enlarged scale, of a handle of the control assembly identified with letter “A” in FIG. 8.

FIG. 10 is an isometric view of an alternative, walking stick embodiment of the present invention in the contracted condition.

FIG. 11 is an isometric view of the embodiment of the present invention illustrated in FIG. 10 in the extended condition.

FIG. 12 is an isometric view of an alternative embodiment of the present invention in the contracted condition.

FIG. 13 is an isometric view of the embodiment of the present invention illustrated in FIG. 12 in the extended condition.

FIG. 14 is a sectional view of an alternative embodiment using a screw drive system, without forearm support and in the extended condition.

FIG. 15 is a detailed isometric section view of the handle and control assembly of an alternative embodiment.

FIG. 16 is a sectional view of an alternative embodiment using a screw drive system, without forearm support and in the retracted condition.

FIG. 17 is a detail view of the portion from FIG. 16 identified by Reference letter B.

### DETAILED DESCRIPTION OF THE PREFERRED EMBODIMENT

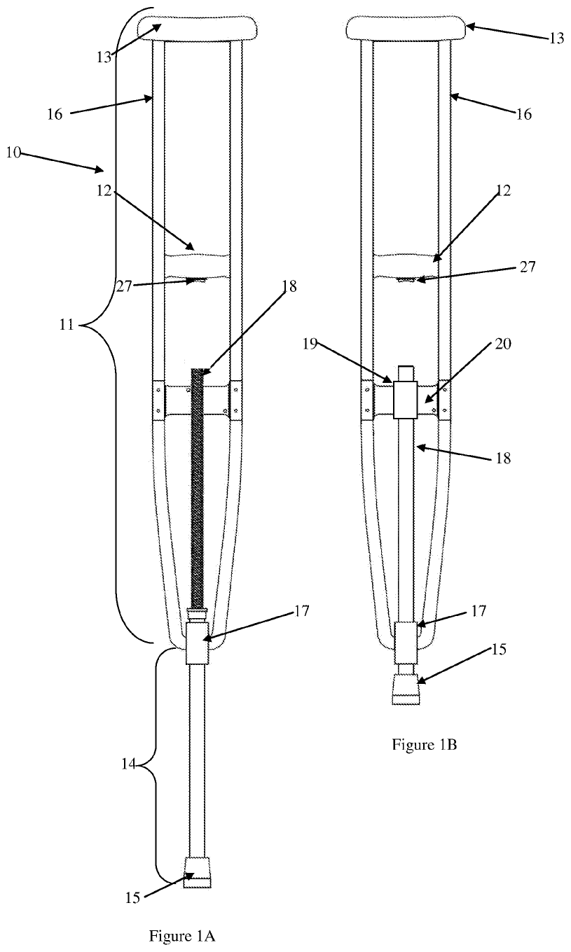

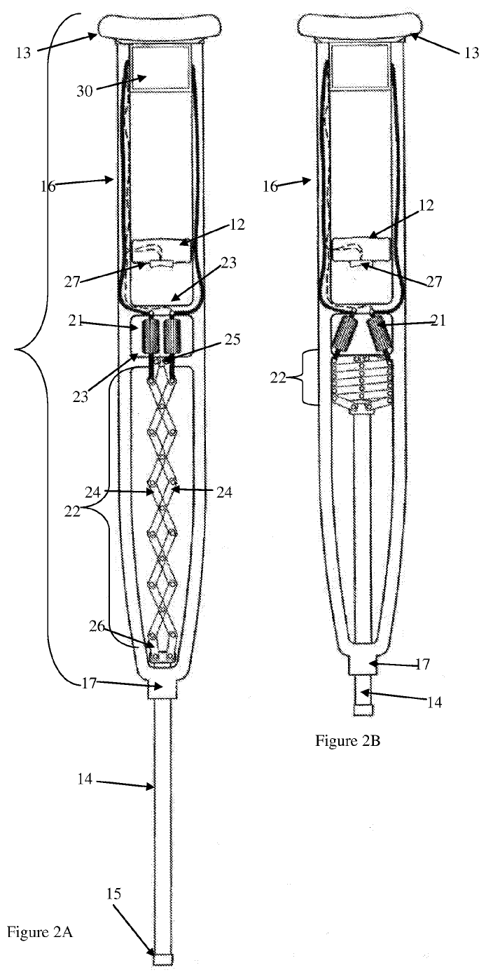

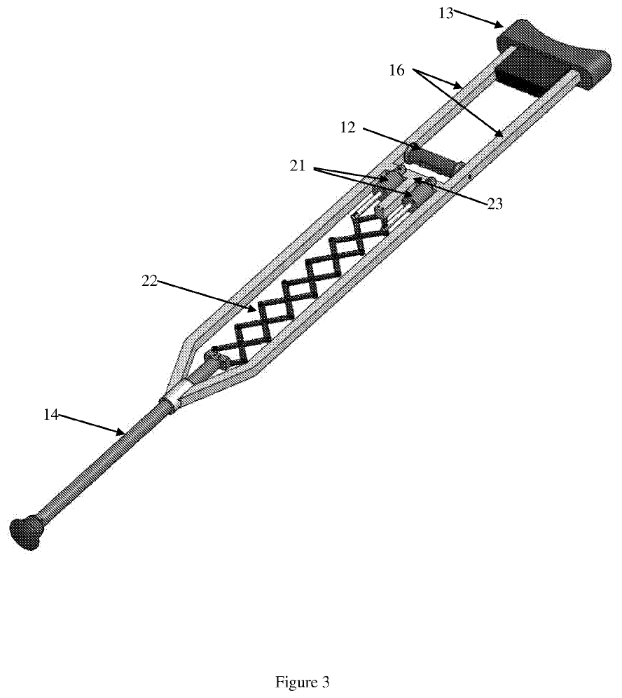

According to a particularly preferred embodiment, an adjustable length crutch is provided. A pair of alternative preferred embodiments illustrated, the first preferred embodiment illustrated in FIGS. 1A and 1B and the second preferred embodiment illustrated in FIGS. 2A, 2B and 3.

Referring firstly to the common features of the crutch as illustrated in all Figures, the adjustable length crutch 10 includes an upper assembly 11 including a hand support 12 and an underarm support 13 to support a portion of the weight of a user, and a leg 14 having a foot 15 adapted to abut the ground in use to balance the user associated upper assembly 11. A length adjustment mechanism is provided to adjust the separation between the supports 12, 13 and the foot 15 to adjust the effective length of the crutch 10.

The upper assembly 11 of the crutch 10 includes a pair of spaced apart struts 16 with the underarm support 13 at an upper end and extending between the struts 16. The underarm support 13 is fixed and also functions to space the struts 16 at the upper end. The hand support 12 extends between the struts 16 approximately at arm's length downwardly from the underarm support 13. The hand support portion will usually be temporarily fixed relative to the struts 16, but the hand support 12 is be movable in order to adjust in the spacing between the hand support 12 and the underarm support 13.

According to this configuration, the struts 16 of the upper assembly 11 taper towards each other at a lower end as illustrated. A guide portion 17 is provided at or adjacent the lower end of the upper assembly 11 in order to receive and/or guide the leg 14.

Turning now to the specific embodiment illustrated in FIGS. 1A and 1B, the length adjustment mechanism has a threaded or helical operation and the components of the length adjustment mechanism therefore adapted for this purpose. In particular, the components of the length adjustment mechanism include a threaded rod 18, an electric drive motor 19 to rotate the threaded rod 18 in both directions, and a telescopic leg 14 associated with the threaded rod 18 and which extends and attracts relative to the upper assembly 11 upon rotation of the threaded rod 18 by the electric motor 19.

The electric drive motor 19 is associated with a guide arm 20 extending laterally between the struts 16 of the upper assembly 11. The electric drive motor 19 is a lightweight motor, preferably one which is powered by batteries 30 which are located in or on the underarm support 13. Activation of the electric drive motor 19 causes rotation of the threaded rod 18.

According to this embodiment, each of the portions of the telescopic leg member 14 are provided with an internally threaded portion (not shown) adapted to engage with the threaded rod 18 in order to extend and retract. Typically, extension of the innermost telescopic member is actuated initially by rotation of the threaded rod 18 relative to the internally threaded portion. Once the innermost telescopic member has reached the limit of its movement, the further rotation of the threaded rod 18 may disengage from the internally threaded portion of the innermost member and continued rotation will then extend the next innermost telescopic member and so on until the telescopic leg member reaches the desired length or the limit of its extension. Retraction of the telescopic leg member will preferably occur in the opposite manner.

According to the second preferred form illustrated in FIGS. 2A, 2B and 3, the length adjustment mechanism includes a pair of hydraulic rams 21 associated with an extendable and retractable scissor drive assembly 22 which is in turn associated with the leg 14. According to this embodiment, the leg 14 is of a fixed length and is extended and retracted to the desired position by the scissor drive assembly 22. The hydraulic rams 21 force the extension and retraction of the scissor drive assembly 22.

Each of the hydraulic rams 21 is mounted in a pivotal fashion to a cross piece 23 provided in association with the upper assembly. The hydraulic rams 21 are each associated with the scissor drive assembly 22.

The scissor drive assembly 22 includes a plurality of pairs of arm members 24, the arm members 24 of each pair connected to each other approximately centrally along their length. Each end of each arm member 24 is then attached to an end of an arm member 24 of an adjacent pair which are similarly connected to each other. The end of each of the uppermost pair of arm members 24 is attached to one of the rams 21 via an intervening pivotably mounted plate 25.

The lowermost pair of arm members 24 is associated with the leg member via intervening members 26.

The rams 21 of this configuration are provided between a pair of cross-piece members 23 which are attached to and interposed between the struts 16 provided as a part of the upper assembly 11.

Again referring to both embodiments, the extension and retraction (and typically the speed of extension and retraction) of the leg 14 is actuated by a depressible or button or switch 27. Normally, the actuation button 27 is associated with the hand support 12 so as to be conveniently located for a user when the crutch is in use.

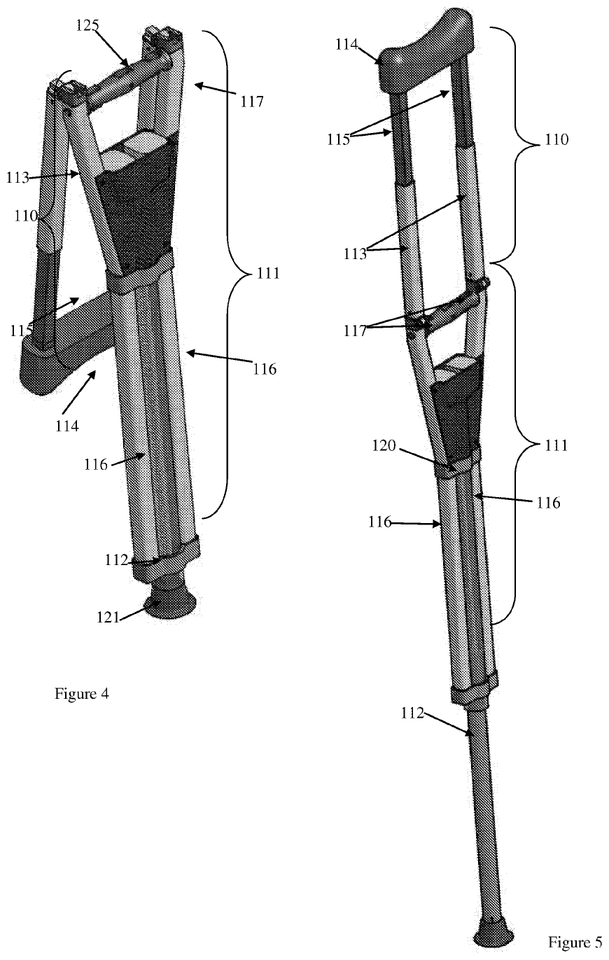

A further embodiment of the crutch of the present invention is illustrated in FIGS. 4 to 9. According to this embodiment, the crutch has frame which is divided into a lower portion 110 and an upper portion 111 which is foldable about a hinge point 118 relative to the lower portion 110 to reduce the effective length of the crutch when in the use condition.

Further, the lower portion has an associated extendable leg 112 which is extendable and retractable relative to the lower portion of the frame. The extendable leg illustrated is provided with a rubber foot 121 in order to minimise slippage.

The upper portion 111 can be locked into at least the use condition in which it is substantially coplanar with the lower portion of hundred and 10 of the frame utilising a locking means which is normally provided at the hinge point 118. According to an alternative embodiment, the upper portion may be complete removal from the lower portion.

The upper portion 111 of the frame has a pair of spaced apart substantially parallel frame members 113. The upper portion is also provided with a shaped shoulder rest 114 which he is adapted to be received in an underarm region when the crutch is used. As illustrated in FIGS. 4 and 5, the frame members 113 each receive an elongate member 115 which is received telescopically within the frame member 113.

The elongate member 115 and the respective frame member 113 can be moved relative to one another in order to adjust the effective length of the upper portion 111 of the frame and are then typically releasably locked to one another to at least temporarily fix the effective length. A variety of methods may be used to lock the members together but typically, a biased pin or button extends outwardly from the elongate member 115 and is received in one of a series of openings spaced over the length of the frame member 113.

The lower portion 110 of the frame includes at least a pair of frame members 116 which are substantially parallel over the majority of the height of the lower portion 110 with a divergent section towards the upper end of the lower portion 110. In practice, a pair of substantially parallel frame members 116 will be provided and a pair of divergent frame members 117 will also be provided attached together using a support 119. A support 119 will also typically be provided at a lower end of the lower portion in order to maintain the separation of the parallel frame members 116.

The hinge point for attachment of the upper portion 111 is located at or adjacent free end of the divergent frame members 117 of the frame members 116.

The lower portion 110 of the frame includes a handle or hand rest 125 at an upper portion thereof. The hand rest typically extends substantially horizontally between the divergent frame is 117 of the frame members 116.

The extendable leg 112 is received between the substantially parallel frame members 116.

The extendable leg 112 has an outer sleeve which is substantially hollow and tubular. The outer sleeve covers and protects the fluid extension mechanism which is best illustrated in FIG. 7.

The preferred embodiment of the fluid extension mechanism includes an extendable and retractable gas strut 120. The gas strut 120 includes a cylinder 122 and a piston including a rod 123 located therein. Between the piston and the cylinder 122 is a quantity of pressurised gas which extends the rod 123 by acting on the piston when the actuated to release the rod 123 and the rod 123 can be pushed back into the cylinder 122 using force applied to the foot 121 of the extendable leg 112 which is coaxially located about the rod 123.

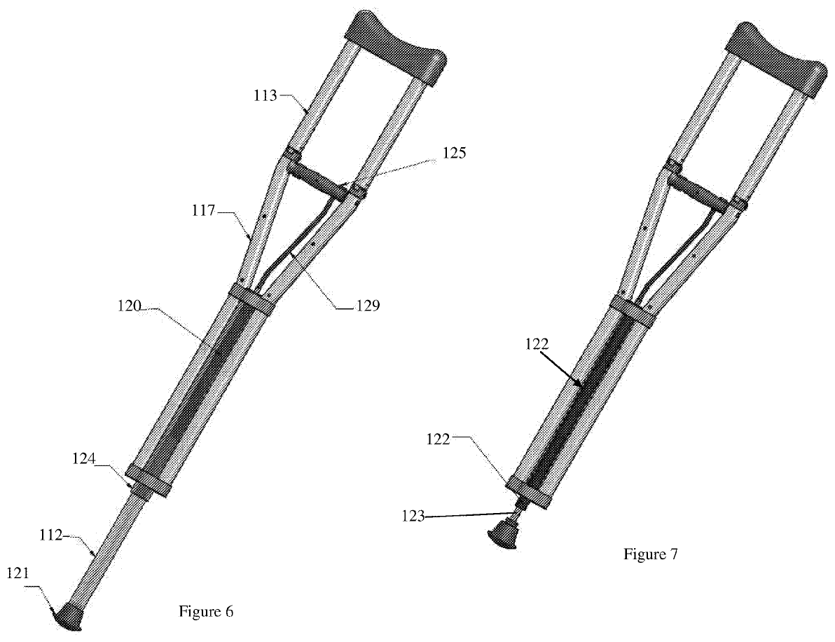

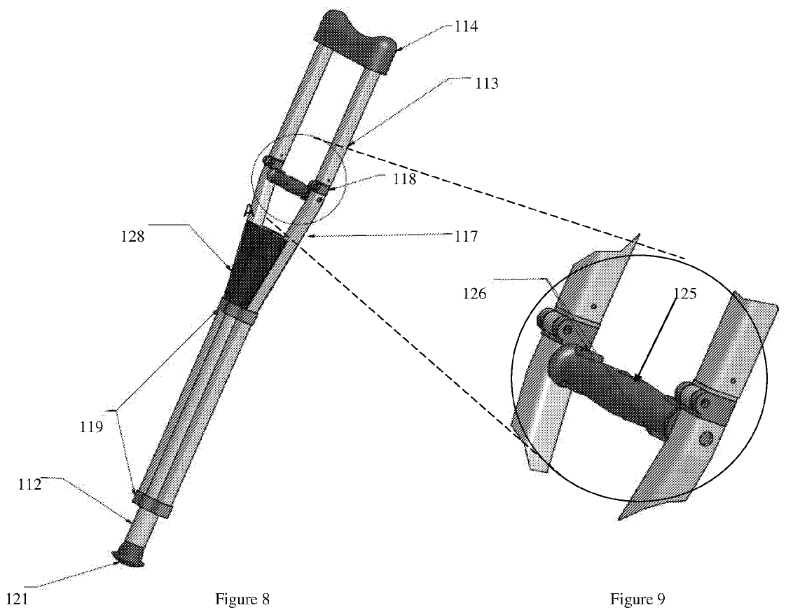

A guide sleeve 124 is provided in the lower support 119 located at the bottom end of the lower portion 111 of the frame. The extendable leg 112 therefore moves between an extended condition illustrated in FIG. 6 and a retracted condition illustrated in FIG. 8.

Controlling the extension and retraction of the gas driven rod is an actuation mechanism which is associated with the hand rest 125. The hand rest 125 is best illustrated in FIG. 9. The hand rest includes a number of actuation buttons adapted to be used to control the extension of the extendable leg 112.

According to the illustrated embodiment, a pair of buttons 126 may be provided on the hand rest 125 at each lateral end of the hand rest 125 and which are movable between a position which actuates extension of the gas strut and a second position which actuates retraction. These buttons 126 are typically rocker buttons and biased into a neutral position between the two actuation positions.

The actuation buttons are connected to the gas strut using a connection conduit 129 which extends from the hand rest 125 to an upper end of the gas strut 120 as illustrated in FIGS. 6 and 7 in particular. The connection conduit 129 is typically protected by a cover 128 which extends on both sides of the crutch between the divergent frame members 117 and also has a closed upper end.

In an alternative embodiment, two actuation buttons are provided and two unlocking buttons are provided on the hand rest 125.

Normally, the unlocking buttons are adapted to unlock the extendable leg 112/rod 123 to allow extension thereof. Typically the unlocking buttons are biased into the locking condition which in turn locks of the extendable leg 112/rod 123 into position (whether extended or retracted). The unlocking buttons typically located on a lower side of the hand rest 125 in order to prevent accidental actuation by a user when placing a load on the upper side of the hand rest 125 which will be the typical location for the load when the crutch is in use.

According to one preferred embodiment, an unlocking button is provided on each lateral end of the hand rest 125 so that the crutch can be used in either the left or right-hand with an unlocking button located adjacent a user's forefinger.

Similarly, a pair of actuation buttons normally provided on or in an upper surface of the hand rest 125 at each lateral end of the hand rest 125 again so that the buttons are adjacent a user's hand in the use condition.

The actuation of the extendable leg 112/rod 123 is therefore preferably a two-part process wherein an unlocking button must be depressed before the actuation button is depressed in order to unlock the extendable leg 112/rod 123 and then actuate the gas strut 120. Typically this same process is required to extend the extendable leg 112/rod 123 as well as to retract the extendable leg 112/rod 123.

As mentioned previously, the preferred gas strut mechanism may be provided with a thread on the rod with a matching thread on an inner surface of the cylinder in order to control the rate of extension and retraction and also to provide the crutch with increased load-bearing capabilities.

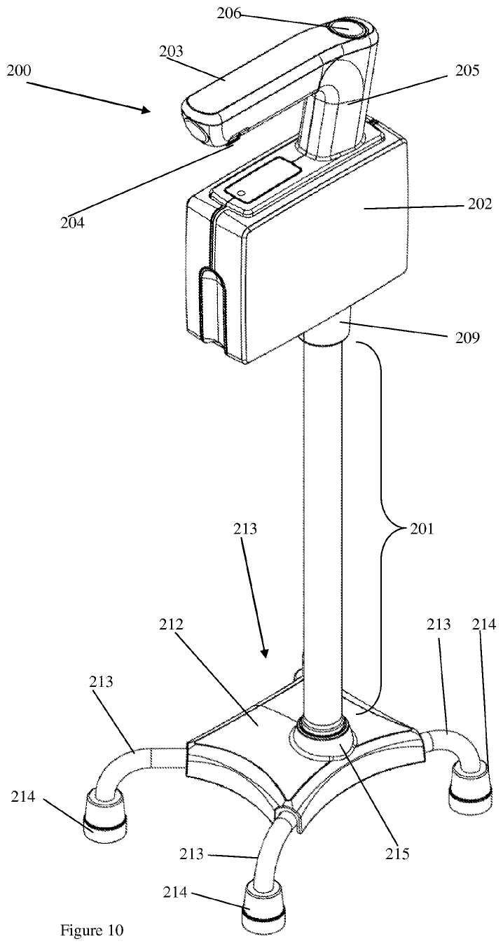

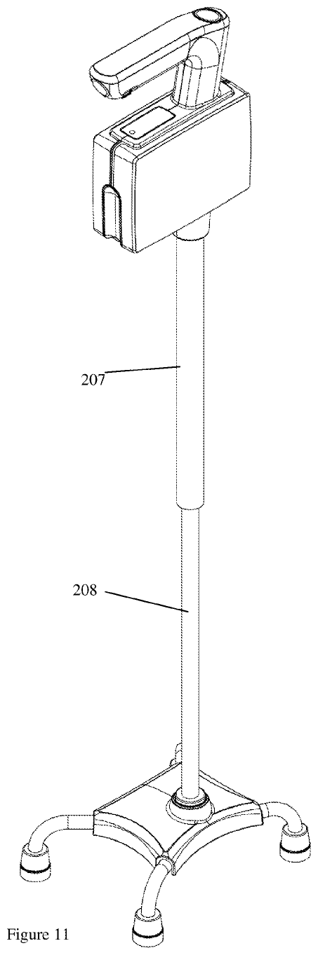

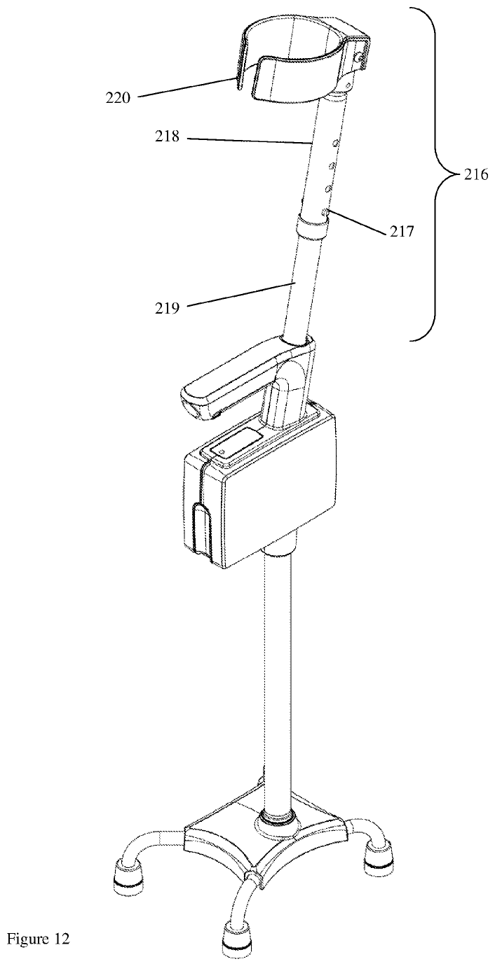

According to a further preferred embodiment illustrated in FIGS. 10 to 13, the crutch includes a gripping portion 200 and a length adjustable leg 201. The leg is extended to an extended position from a retracted position thereby adjusting the length of the crutch. A mechanism which causes the leg portion to extend and a control assembly through which the mechanism can be operated are provided in a control housing 202.

The gripping portion 200 of the crutch of the embodiments illustrated in FIGS. 10 to 13 is substantially L-shaped with a handle 203 to be received and held by a user, in the user's palm with the user's fingers and thumb wrapped thereabouts.

In use, the handle is located relative to a user's palm such that the handle can support the thenar and hypothenar eminences of the user's hand with the user's fingers located underneath the handle portion on one side of the handle portion and user's thumb wrapped about the handle portion from the opposite side of the handle portion. The handle portion will therefore be shaped accordingly.

An actuator switch 204 is located on an underside of the handle 203 recessed into the handle in order to prevent accidental actuation. The actuator switch is located at a forward or free end of the handle 203 in order to be operated by a user's forefinger or thumb.

The actuator switch of the illustrated embodiment is a toggle switch biased into a central, inactive position and which is capable of movement to either or both of the left and right actuation positions. Movement of the toggle switch to the left (all right) will actuate extension of the crutch and movement of the toggle switch in the opposite direction will allow retraction. In the retraction position, the toggle switch simply unlocks the leg allowing a user to apply force to the leg in order to force retraction.

The opposite end of the handle 203 is provided with a depending portion 205. As illustrated in FIGS. 10 to 13, the depending portion is angled relative to the handle 203 at an angle slightly less than 90°.

The depending portion 205 is at least partially hollow with an opening 206 provided into the depending portion 205 substantially coaxial with the depending portion 205 through the upper surface of the handle 203. A cover member is provided for this opening.

The leg portion 201 of the embodiment illustrated in FIGS. 10 to 13 is provided as a telescoping upper, outer leg portion 207 and lower inner leg portion 208.

According to a preferred embodiment illustrated in FIGS. 10 to 13, a control housing 202 is provided between the gripping portion 200 and the leg portion 201 with the upper leg portion 207 is attached to a lower side of the control housing 202 allowing the mechanism within the control housing 202 to drive extension of the leg portion 201. Preferably, the upper leg portion is received at least partially within an annular mounting collar 209.

The lower leg portion can be provided with a shock absorber 210 at a lower end of the lower leg for absorption of shock when the leg portion 201 is extended and the crutch is in use, or alternatively, to buffer the final movement of the lower leg 208 wherein the lower leg is being retracted.

A foot 211 is provided at the lower end of the lower leg portion 208. In the preferred embodiment, the foot includes a foot platform 212 with a large effective area and a plurality of arms 213 with non-slip rubber caps 214 extending laterally and then downwardly from the foot platform 212.

The extendable leg portion 201 is mounted to one side of the foot platform 212, adjacent one side edge using a ball joint 215 allowing support to be provided closer to the user's body then if the leg portion 201 was more centrally mounted on the platform 212.

In this embodiment, the crutch includes a mechanism to cause the leg portion to move to the extended position from a retracted position thereby adjusting length of the crutch. The control mechanism is typically contained within a control housing 202 which is a central component of the crutch located typically relative to the gripping portion 200 as discussed above.

The control housing 202 is generally rectangular, extending forwardly beneath the handle 203 of the gripping portion 200 approximately as far as the free end of the handle 203. Accordingly, the length of the control housing is preferably coaxial with the handle portion and located beneath the handle portion. Typically, the control housing is of minimal width, again approximating the width of the handle portion.

The control housing typically houses the actuator to cause extension of the leg such that when the actuator switches operated into either the extension or retraction position, this allows a drive system to move or drive the movement of the leg as required.

Typically, the drive system is either hydraulic or pneumatic. Therefore, when the actuator switch is switched to the extension position, electrical power provided from a preferred the battery (which can be rechargeable or replaceable) within the control housing is provided to the pump in order to drive a fluid to extend the lower leg relative to the upper leg.

Once the limit of extension is reached, a lock (not shown) can preferably engage either automatically will be engaged manually by the user. Once the lock has been engaged, manual disengagement is preferably preferred.

When the actuator is switched to the retraction position, then the pump can either operate in reverse at a given (adjustable) speed to reduce the length of the crutch at a controlled speed or in a controlled manner or alternatively, the lock will be disengaged and the user can be required or able to exert force on the handle portion to drive retraction of the leg.

The crutch of the preferred embodiment may include an optional support 216 which is attachable to the crutch to provide additional support stability. The support will normally be attached utilising an extension portion with one end of the extension portion attached into the opening 206 in the handle 203 of the gripping portion 200 (once the cover has been removed). Typically, a part of the extension portion will extend down into the depending portion of the gripping portion for stability.

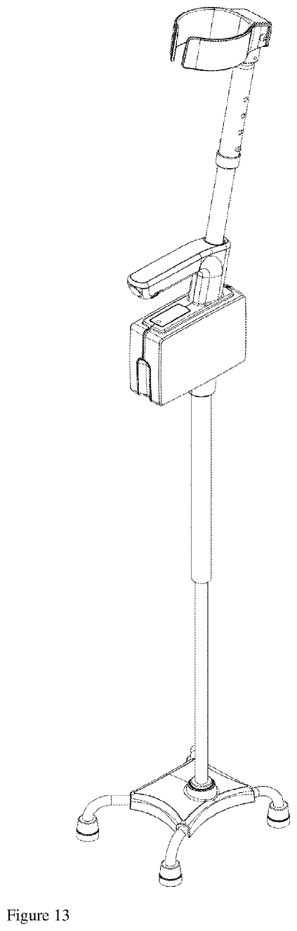

The extension portion illustrated in FIGS. 12 and 13 is a length adjustable multipart portion. Length adjustment of this portion is provided in the form of a pin 217 for location in aligned or alignable openings in the upper 218 and lower 219 telescoping components of the extension portion.

The extension portion illustrated in FIGS. 122 and 13 is provided with a collar or cuff 220 at an upper end to receive a user's forearm or alternatively, a pad or similar could be provided to be received in a user's armpit area.

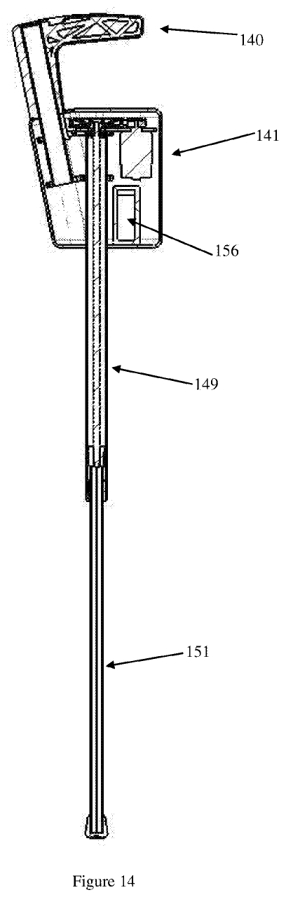

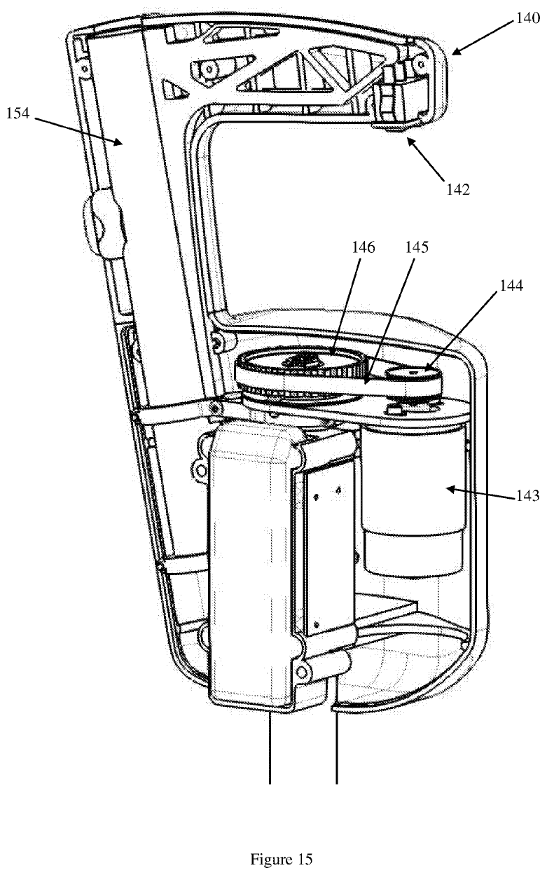

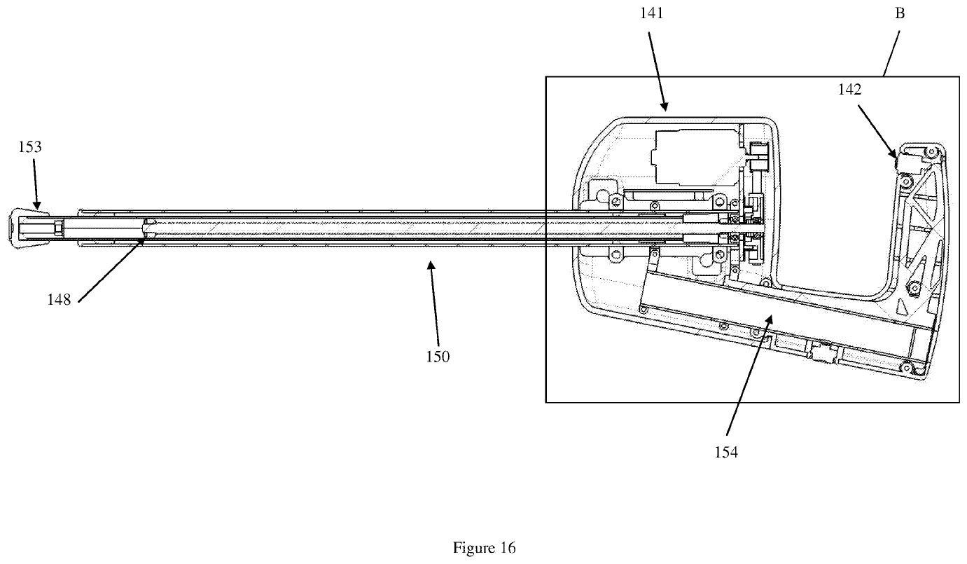

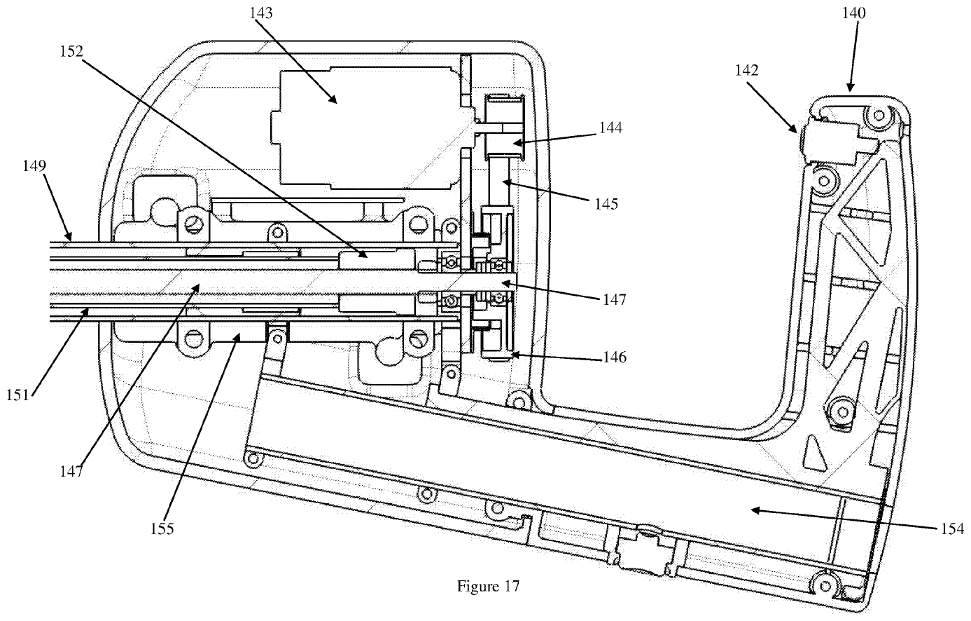

In an alternative form illustrated in FIGS. 14 to 17, the drive system can be a screw drive system. In a preferred embodiment, the handle 140 and control assembly 141 are integrated into a single housing and the handle 140 includes a toggle switch 142 with an “extend” position and a “retract” position on either side of a home or “locked” position. Operation of the toggle switch 142 activates an onboard electrical motor 143 to rotate a drive cog 144 in the desired direction. The motor is typically powered by a rechargeable on board battery 156. The drive cog 144 mounts a closed loop drive belt 145, preferably a toothed drive belt. A driven cog 146 is provided adjacent to the drive cog 144 and separated therefrom but connected by the drive belt 145. As illustrated in FIGS. 25 to 17, the driven cog 146 is larger in diameter than the drive cog 144 which increases the torque available at the driven cog 146. The driven cog 146 is also a toothed cog.

As shown in FIG. 17 in particular, the driven cog 146 rotates an upper portion of a threaded rod 147 which is a part of an extendible leg assembly 150. The upper portion of the threaded rod 147 is not threaded but the bulk of the length of the threaded rod 147 is threaded. A lower portion of the threaded rod 147 is provided with a guide 148 which is fixed to the threaded rod 147 and abuts an inner surface of a hollow leg tube 149.

The threaded rod 147 is mounted within a hollow leg tube 149 of fixed length which extends from the control assembly 141. A hollow extendible leg 151 is mounted about the threaded rod 147 and within the hollow leg tube 149. The hollow leg tube 149 is made of metal and is circular in cross-section.

The hollow extendible leg 149 is provided with a guide boss 152 at an upper end that is mounted about the threaded portion of the threaded rod 147 and at least a portion of the guide boss 152 abuts an inner surface of the hollow leg tube 149. A threaded bore is provided through the guide boss 152 to engage with the threaded portion of the threaded rod 147. Rotation of the threaded rod 147 will therefore preferably cause translation of the guide boss 152 along the threaded rod 147 which extends and retracts the hollow extendible leg 151, which in this embodiment illustrated, has a rubber or similar foot 153 (rather than the four legged assembly illustrated earlier but which can be used).

A receiving tube 154 is also preferably provided in the handle 140 accessible using the opening described above. When the preferred cover is removed, a lower end of a forearm support 216 can be inserted into the receiving tube 154. As shown in FIG. 17 best, he receiving tube 154 is attached to a housing 155 that is attached to the hollow leg tube 149 and surrounds the guide boss 152 and the upper end of the drive mechanism. The handle portion of the device is also attached to the receiving tube 154. The receiving tube 154 is preferably made of metal in order to provided additional strengthening and rigidity to the device allow significant forces to be transmitted to the leg assembly 150.

In the present specification and claims (if any), the word “comprising” and its derivatives including “comprises” and “comprise” include each of the stated integers but does not exclude the inclusion of one or more further integers.

Reference throughout this specification to “one embodiment” or “an embodiment” means that a particular feature, structure, or characteristic described in connection with the embodiment is included in at least one embodiment of the present invention. Thus, the appearance of the phrases “in one embodiment” or “in an embodiment” in various places throughout this specification are not necessarily all referring to the same embodiment. Furthermore, the particular features, structures, or characteristics may be combined in any suitable manner in one or more combinations.

1. An adjustable length crutch including at least one support portion to support at least a portion of the weight of a user, a leg portion associated with the at least one support portion and having a foot adapted to abut the ground in use to balance the user and a length adjustment mechanism to adjust the separation between the at least one support portion and the foot to adjust the effective length of the crutch.

2. An adjustable length crutch as claimed in claim 1 wherein the length adjustment mechanism includes a screw drive system to adjust the effective length of an extendible leg assembly.

3. An adjustable length crutch as claimed in claim 2 wherein the length adjustment mechanism includes a toggle switch with an extend position and a retract position on either side of a home or locked position, operation of the toggle switch activating the screw drive system.

4. An adjustable length crutch as claimed in claim 2 wherein the screw drive mechanism includes an onboard electrical motor.

5. An adjustable length crutch as claimed in claim 4 wherein the screw drive mechanism includes a drive cog driven rotatably by the electrical motor, a closed loop drive belt, and driven cog provided adjacent to the drive cog and separated therefrom, connected by the drive belt.

6. An adjustable length crutch as claimed in claim 5 wherein the driven cog is larger in diameter than the drive cog which will increase the torque available at the driven cog.

7. An adjustable length crutch as claimed in claim 5 wherein the drive cog and the driven cog are both toothed cogs and a toothed drive belt is provided.

8. An adjustable length crutch as claimed in claim 5 wherein the driven cog rotates an upper portion of a threaded rod.

9. An adjustable length crutch as claimed in claim 8 wherein a lower portion of the threaded rod is provided with a guide which is fixed to the threaded rod.

10. An adjustable length crutch as claimed in claim 5 wherein the screw drive mechanism includes a threaded rod mounted within a hollow leg tube of fixed length with a hollow extendible leg mounted about the threaded rod and within the hollow leg tube.

11. An adjustable length crutch as claimed in claim 10 wherein the hollow extendible leg is provided with a guide boss at an upper end that is mounted about the threaded rod and at least a portion of the guide boss abuts an inner surface of the hollow leg tube.

12. An adjustable length crutch as claimed in claim 11 wherein a bore is provided through the guide boss and the bore is threaded to engage with the threaded rod so that rotation of the threaded rod causes translation of the guide boss along the threaded rod which extends and retracts the hollow extendible leg.

13. An adjustable length crutch as claimed in claim 1 further including a receiving tube provided in the at least one support portion into which a lower end of a forearm support is insertable.

14. An adjustable length crutch as claimed in claim 13 wherein the receiving tube is mounted relative to the leg portion.

15. An adjustable length crutch as claimed in claim 10 wherein the speed at which extension occurs is adjustable and similarly, the speed at which retraction occurs may be adjustable as well by providing a threaded portion of a different pitch at an lower and/or upper end of the threaded rod.

16. An adjustable length crutch as claimed in claim 1 further including an alarm actuator with an associated communications pathway to allow a user of the crutch to alert others in an emergency.

17. An adjustable length crutch as claimed in claim 16 wherein the actuator is a button or switch or similar and actuation causes an alarm to be issued or to transmit a signal to a remote location.

18. An adjustable length crutch as claimed in claim 16 wherein the crutch is provided with a unique identifier allowing identification of the user if the actuator is triggered.

19. An adjustable length crutch as claimed in claim 16 wherein the crutch is provided with a positioning device in order that the location of the crutch can be ascertained at any time.

20. An adjustable length crutch as claimed in claim 1 further including an attitude sensing device to actuate an alarm or signal should the crutch be placed in an angle that indicates that the person has fallen.

21. An adjustable length crutch as claimed in claim 20 wherein a manual deactivation of the alarm or signal is provided such that the operator can disable the attitude sensor trigger as required.
